(19) United States
(12) Patent Application Publication
(10) Pub. No.: US 2011/0118362 A1
(43) Pub. Date: May 19, 2011

(54) POLYMERS FOR CARDIOPULMONARY THERAPIES

(76) Inventors: Randal Dull, (US); Kristina Giantsos, (US)

(21) Appl. No.: 12/618,317

(22) Filed: Nov. 13, 2009

(51) Int. Cl. A61K 47/32; C08F 226/02; C08F 220/60; C08F 12/30; A61P 11/00

(52) U.S. Cl. 514/772.4; 526/307.2; 526/307; 526/243

(57) ABSTRACT

Disclosed are compositions containing copolymers with hydroxypropyl methacrylamide and methods of using the compositions to treat cardiopulmonary diseases are also disclosed.

### CROSS REFERENCE TO RELATED APPLICATIONS

This application claims the benefit of priority to U.S. Provisional Application No. 61/114,743, filed Nov. 14, 2009, which is incorporated by this reference herein in its entirety.

### ACKNOWLEDGEMENT OF GOVERNMENT SUPPORT

This invention was made with government support under Grant No. 59306690 awarded by the National Institutes of Health. The government has certain rights in the invention.

### BACKGROUND

Acute changes in lung capillary permeability continue to complicate procedures such as cardio-pulmonary bypass, solid organ transplant, and major vascular surgery and precipitates the more severe disease state Adult Respiratory Distress Syndrome (ARDS), affecting 64.2 patients per 100,000 per year. (Goss, C. H., Brower, R. G., Hudson, L. D., Rubenfeld, G. D. “Incidence of acute lung injury in the United States,” Crit. Care Med. 31(6):1607-11, 2003.) To date there is no treatment targeted directly to the lung microvasculature. What are needed are compositions and methods for the treatment of such cardiopulmonary indications.

### SUMMARY

In accordance with the purposes of the disclosed materials, compounds, compositions, articles, and methods, as embodied and broadly described herein, the disclosed subject matter, in one aspect, relates to compositions and methods for preparing and using such compositions. In a further aspect, the disclosed subject matter relates to compositions of polymers. The disclosed compositions can be infusible. The disclosed compositions can contain hydroxypropyl methacrylate and/or tetramethyl ammonium chloride moieties. Methods of making and using such compositions are also disclosed.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE FIGURES

The accompanying figures, which are incorporated in and constitute a part of this specification, illustrate several aspects described below.

FIG. 1 is a pair of graphs showing cytotoxicity of copolymers as ascertained through LDH release for (A) P-AP and (B) P-TMA Cl of increasing concentrations. Copolymers containing primary amines showed significant cytotoxicity at higher mol % composition and higher concentration, whereas the cytotoxicity of quaternary amine copolymers exceeded 10% only at maximal amine mol % composition and concentration.

FIG. 2 is a pair of confocal images of surface bound 40P-(FITC)TMA Cl and actin cytoskeleton as seen at (A) 45° and (B) 90° to the z axis. Figure incorporates approximately 75 cells imaged at 40× magnification. 40P-(FITC)-TMA covers the monolayer after vigorous rinsing steps and actin staining demarcates the cell membrane from the cytoplasmic compartment.

FIG. 3 is a group of graphs showing permeability coefficients of albumin diffusion across confluent BLMVEC in the presence of (A) 40P-TMA Cl or (B) P-HPMA, 40P-TMA Cl reduces P_{DA} in a concentration dependant manner consistent with quantitative binding to the glycocalyx and enhancement of passive barrier function. P-HPMA was unable to significantly reduce enhancement of passive barrier function. P-HPMA was unable to significantly reduce P_{DA} with respect to control at any concentration. (C) Addition of bradykinin to monolayers increases permeability coefficient and addition of 40P-TMA-Cl abolishes the bradykinin-induced increase in P_{DA}. *P<0.05; Nonparametric Dunn's test, N=3.

FIG. 4 is a graph outline for polymer application and pressure increases during an Lₚ experiment as a factor of time.

FIG. 5 is a graph of a MTT assay for cell viability (%) at 20, 40, and 80 mol % P(TMA) as compared to HPMA. Error bars are ±SD of 3 replicates.

FIG. 6 is a graph of hydraulic conductivity measurement taken from control cells (stripe) and cells treated with P-HPMA (Black) or 40P-TMA-Cl (shaded). 40P-TMA-Cl significantly reduced peak normalized Lₚ at 20 cm H₂O pressure. *P<0.05; Dunn's nonparametric test, N=5-7.

### DETAILED DESCRIPTION

The materials, compounds, compositions, and methods described herein may be understood more readily by reference to the following detailed description of specific aspects of the disclosed subject matter, the Figures, and the Examples included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General and Chemical Definitions

In this specification and in the claims that follow, reference will be made to a number of terms, which shall be defined to have the following meanings.

Throughout the specification and claims the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a compound” includes mixtures of two or more such compounds, reference to “an agent” includes mixtures of two or more such agents, reference to “the composition” includes mixtures of two or more such compositions, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. The term “about” means within 5% of the stated value. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value,” and possible ranges between values are also disclosed, as appropriately understood by the skilled artisan. For example, if the value “10” is disclosed, then “less than or equal to 10” as well as “greater than or equal to 10” is also disclosed. It is also understood that throughout the application data are provided in a number of different formats and that these data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

References in the specification and concluding claims to parts by weight of a particular component in a composition denotes the weight relationship between the component and any other components in the composition for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, a “mole percent” or “mol %” of a component, unless specifically stated to the contrary, refers to the ratio of the number of moles of the component to the total number of moles of the composition in which the component is included, expressed as a percentage.

“Contacting” means an instance of exposure by close physical contact of at least one substance to another substance.

“Admixture,” “mixture,” or “blend” is generally used herein to refer to a physical combination of two or more different components. In the case of polymers, an admixture, mixture, or blend of polymers is a physical blend or combination of two or more different polymers. Admixtures can, though need not, result in a reaction between the admixed components, resulting in a composition where little or none of the original components are present.

“Sufficient amount” and “sufficient time” mean an amount and time needed to achieve the desired result or results, e.g., dissolve a portion of the polymer.

“Biocompatible” as used herein refers to a material that is generally non-toxic to the recipient and does not possess any significant adverse effects to the subject and, further, that any metabolites or degradation products of the material are non-toxic to the subject.

“Biodegradable” refers to a material that will degrade or erode under physiologic conditions to smaller units or chemical species and are capable of being metabolized, eliminated, or excreted by the subject.

The general term “polymer” includes homopolymer, copolymer, etc. unless the context clearly dictates otherwise.

“Molecular weight” as used herein, unless otherwise specified, refers to the relative average chain length of the bulk polymer. In practice, molecular weight can be estimated or characterized in various ways including gel permeation chromatography (GPC) or capillary viscometry. GPC molecular weights are reported as the weight-average molecular weight (Mw) as opposed to the number-average molecular weight (Mn). Capillary viscometry provides estimates of molecular weight as the inherent viscosity determined from a dilute polymer solution using a particular set of concentration, temperature, and solvent conditions.

“Controlled release” as used herein means the use of a material to regulate the release of another substance.

“Bioactive agent” is used herein to include a compound of interest contained in the disclosed compositions, such as therapeutic or biologically active compounds. It includes without limitation physiologically or pharmacologically active substances that act locally or systemically in the body. A biologically active agent is a substance used for, for example, the treatment, prevention, diagnosis, cure, or mitigation of disease or illness, a substance which affects the structure or function of the body, or pro-drugs, which become biologically active or more active after they have been placed in a predetermined physiological environment. Bioactive agents include biologically, physiologically, or pharmacologically active substances that act locally or systemically in the human or animal body. Examples can include, but are not limited to, drugs, small-molecule drugs, vaccines, adjuvants, peptides, proteins, nucleic acids, nucleotides, and oligonucleotides. “Bioactive agent” includes a single such agent and is also intended to include a plurality of bioactive agents including, for example, combinations of two or more bioactive agents.

“Excipient” is used herein to include any other compound that can be contained in the disclosed compositions that is not a therapeutically or biologically active compound. As such, an excipient should be pharmaceutically or biologically acceptable or relevant, for example, an excipient should generally be non-toxic to the subject. “Excipient” includes a single such compound and is also intended to include a plurality of excipients.

“Agent” is used herein to refer generally to compounds that are contained in or on a composition. Agent can include a bioactive agent or an excipient. “Agent” includes a single such compound and is also intended to include a plurality of such compounds.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen and oxygen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. Also, as used herein “substitution” or “substituted with” is meant to encompass configurations where one substituent is fused to another substituent. For example, an aryl group substituted with an aryl group (or vice versa) can mean that one aryl group is bonded to the second aryl group via a single sigma bond and also that the two aryl groups are fused, e.g., two carbons of one alkyl group are shared with two carbons of the other aryl group.

“A¹,” “A²,” “A³,” and “A⁴” are used herein as generic symbols to represent various specific substituents. These symbols can be any substituent, not limited to those disclosed herein, and when they are defined to be certain substituents in one sentence it does not mean that, in another sentence, they cannot be defined as some other substituents.

The term “alkyl” as used herein is a branched or unbranched saturated hydrocarbon group of 1 to 40 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, octadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, sulfo-oxo, sulfonylamino, nitro, silyl, azide, nitro, nitrile, or thiol, as described below. A “lower alkyl” is an alkyl group with up to six carbon atoms, e.g., methyl, ethyl, propyl, butyl, pentyl, and hexyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group.

For example, the term “alkyl halide” specifically refers to an alkyl group that is substituted with one or more halides, e.g., fluorine, chlorine, bromine, or iodine. When “alkyl” is used in one sentence and a specific term such as “alkyl halide” is used in another, it is not meant to imply that the term “alkyl” does not also refer to specific terms such as “alkyl halide” and the like.

This practice is also used for other groups described herein. That is, while a term such as “heteroaryl” refers to both unsubstituted and substituted heteroaryl moieties, the substituted moieties can, in addition, be specifically identified herein; for example, a particular substituted heteroaryl can be referred to as, e.g., an “alkyl heteroaryl.” Similarly, a substituted alkenyl can be, e.g., an “alkenyl halide,” and the like. Again, the practice of using a general term, such as “heteroaryl,” and a specific term, such as “alkyl heteroaryl,” is not meant to imply that the general term does not also include the specific term.

The term “alkoxy” as used herein is an alkyl or cycloalkyl group bonded through an ether linkage; that is, an “alkoxy” group can be defined as —OA¹ where A¹ is alkyl or cycloalkyl as defined above. A “lower alkoxy” is an alkoxy group with up to six carbon atoms, e.g., methoxy, ethoxy, propoxy, butoxy, pentoxy, and hexoxy.

The term “alkenyl” as used herein is a hydrocarbon group of from 2 to 40 carbon atoms with a structural formula containing at least one carbon-carbon double bond. Asymmetric structures such as (A¹A²)C═C(A³A⁴) are intended to include both the E and Z isomers. This may be presumed in structural formulae herein wherein an asymmetric alkene is present, or it may be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, sulfo-oxo, sulfonylamino, nitro, silyl, azide, nitro, nitrile, or thiol.

The term “alkynyl” as used herein is a hydrocarbon group of 2 to 40 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, sulfo-oxo, sulfonylamino, nitro, silyl, azide, nitro, nitrile, or thiol.

The term “aryl” as used herein is a group that contains any carbon-based aromatic group including, but not limited to, benzene, benzyl, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, sulfo-oxo, sulfonylamino, azide, nitro, nitrile, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, cyclooctyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, sulfo-oxo, sulfonylamino, nitro, azide, nitrile, silyl, or thiol.

The term “cycloalkenyl” as used herein is a non-aromatic carbon-based ring composed of at least three carbon atoms and contains at least one double bound, e.g., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, sulfo-oxo, sulfonylamino, nitro, silyl, azide, nitrile, or thiol.

The term “cyclic group” is used herein to refer to either aryl groups (e.g., heteraryl, biaryl), non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula:

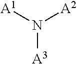

where A¹, A², and A³ can each be, independent of one another, hydrogen, an alkyl, halogenated alkyl, alkenyl, alkynyl, aryl, heteroaryl, cycloalkyl, cycloalkenyl, heterocycloalkyl, or heterocycloalkenyl group described above. Also, any of the A¹, A², and A³ substituents can be absent and any of the remaining substituents can be a multivalent group, i.e., form more than one bond with N.

The term “carboxylic acid” as used herein is represented by the formula —C(O)OH. The term “carboxylate” is a carboxylic acid that has been deprotonated, —C(O)O—. Protonation and deprotonation can be achieved by changes in pH. The terms “carboxylic acid” and “carboxylate” are understood to be interchangeable.

The term “ester” as used herein is represented by the formula —OC(O)A¹ or —C(O)OA¹, where A¹ can be a substituted or unsubstituted alkyl, cycloalkyl, alkenyl, cycloalkenyl, alkynyl, cycloalkynyl, aryl, or heteroaryl group as described herein.

The term “ether” as used herein is represented by the formula A¹OA², where A¹ and A² can be, independently, a substituted or unsubstituted alkyl, cycloalkyl, alkenyl, cycloalkenyl, alkynyl, cycloalkynyl, aryl, or heteroaryl group described herein.

The term “halide” as used herein refers to the halogens fluorine, chlorine, bromine, and iodine.

The term “hydroxyl” as used herein is represented by the formula —OH.

The term “ketone” as used herein is represented by the formula A¹(O)A², where A¹ and A² can be, independently, a substituted or unsubstituted alkyl, cycloalkyl, alkenyl, cycloalkenyl, alkynyl, cycloalkynyl, aryl, or heteroaryl group as described herein.

The term “azide” as used herein is represented by the formula —N₃.

The term “nitro” as used herein is represented by the formula —NO₂.

The term “nitrile” as used herein is represented by the formula —CN.

The term “silyl” as used herein is represented by the formula —SiA¹A²A³, where A¹, A², and A³ can be, independently, hydrogen or a substituted or unsubstituted alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or a substituted or unsubstituted alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or a substituted or unsubstituted alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, a substituted or unsubstituted alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, a substituted or unsubstituted alkyl, cycloalkyl, alkenyl, cycloalkenyl, alkynyl, cycloalkynyl, aryl, or heteroaryl group as described herein.

The term “sulfonamide” as used herein is represented by the formula —S(O)₂NA¹-, where A¹ can be hydrogen, a substituted or unsubstituted alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” and “Rⁿ,” where n is some integer, as used herein can, independently, possess two or more of the groups listed above. For example, if R is a straight chain alkyl group, one of the hydrogen atoms of the alkyl group can optionally be substituted with a hydroxyl group (OH), an alkoxy group, halide, etc. Depending upon the groups that are selected, a first group can be incorporated within second group or, alternatively, the first group can be pendant (i.e., attached) or fused to the second group.

The terms “ortho,” “meta,” and “para” refer to 1,2-, 1,3-, and 1,4-disubstituted benzenes, respectively.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixtures.

It is understood that polymers are referenced herein by referring to the particular monomers that are used to make up the polymer. The monomers are, of course, not actually present in the polymer, except perhaps for some residual amount left over from the polymerization reaction. So, for example, polymethylmethacrylate does not actually contain methylmethacrylate (again except perhaps for some residual unreacted monomer); it contains repeating units from the polymerized monomer methylmethacrylate. This naming convention is common in the art.

Reference will now be made in detail to specific aspects of the disclosed materials, compounds, compositions, components, devices, articles, and methods, examples of which are illustrated in the following description and examples, and in the figures and their previous and following description.

### Materials and Compositions

Disclosed herein are materials, compounds, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a compound is disclosed and a number of modifications that can be made to a number of components or residues of the compound are discussed, each and every combination and permutation that are possible are specifically contemplated unless specifically indicated to the contrary. Thus, if a class of components A, B, and C are disclosed as well as a class of components D, E, and F and an example of a combination composition A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific aspect or combination of aspects of the disclosed methods, and that each such combination is specifically contemplated and should be considered disclosed.

Disclosed herein are compositions of polymers for cardiopulmonary therapies. For example, the disclosed compositions comprise copolymers of N-2-hydroxypropyl methacrylamide (HPMA) with either methylacrylamidopropyl trimethyl ammonium chloride (MA-TMA-Cl) or N-(3-aminopropyl)methacrylamide (MA-AP). Such copolymers optionally including 5-3-(methacryloylaminopropyl)thioureidyl fluorescein (MA-FITC) residues are also disclosed. The disclosed polymers possess positive charges and are disclosed herein as an infusible therapy to target the negatively charged lung capillary glycocalyx in order to mechanically enhance the capillary barrier and turn off mechanotransduction.

The copolymers disclosed herein can be prepared by methods known in the art and involve copolymerizing HPMA and either MA-TMA-Cl or MA-AP, optionally with MA-FITC. The copolymers can contain varying amounts of HPMA, MA-TMA-Cl, MA-AP, and or MA-FITC residues. The substitution and nomenclature herein refers to the number of side chains having propyl-trimethylammonium chloride (“P-TMA-Cl”) or propyl aminopropyl (“P-AP”) substituents. It is also contemplated that the monomer MA-TMA can have other counterions besides chloride, e.g., F⁻, Br⁻, I⁻, SO₄²⁻, CO₂⁻, OH⁻, CH₃CO₂⁻, and the like.

The copolymers can be represented by the molar ratio of HMPA to MA-TMA-Cl or HMPA to MA-AP in the copolymerization reaction. For example, the molar ratio of methylacrylamide to methylacrylamidopropyl trimethyl ammonium chloride can be in the range of from about 9 to 1 to about 6 to 4. In the disclosed copolymers, HPMA can be present in an amount of from about 50 to about 90 mole percent. For example, the lactide can be present in an amount of from about 50 to about 80 mole percent, from about 50 to about 70 mole percent, from about 50 to about 60 mole percent, from about 60 to about 90 mole percent, from about 60 to about 80 mole percent, from about 60 to about 70 mole percent, from about 70 to about 90 mole percent, from about 70 to about 80 mole percent, from about 80 to about 90 mole percent, from about 55 to about 85 mole percent, from about 55 to about 75, from about 55 to about 65, or from about 80 to about 60 mole percent. In other examples, lactide can be present in about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 mole percent, where any of the stated values can form an upper or lower endpoint of a range.

In the disclosed copolymers, MA-TMA-Cl can be present in an amount of from about 10 to about 50 mole percent. For example, the lactide can be present in an amount of from about 10 to about 40 mole percent, from about 10 to about 30 mole percent, from about 10 to about 20 mole percent, from about 20 to about 50 mole percent, from about 20 to about 40 mole percent, from about 20 to about 30 mole percent, from about 30 to about 50 mole percent, from about 30 to about 40 mole percent, from about 15 to about 45 mole percent, from about 25 to about 45 mole percent, from about 25 to about 35, from about 15 to about 35, or from about 20 to about 40 mole percent. In other examples, lactide can be present in about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mole percent, where any of the stated values can form an upper or lower endpoint of a range.

In the disclosed copolymers, MA-AP can be present in an amount of from about 10 to about 50 mole percent. For example, the lactide can be present in an amount of from about 10 to about 40 mole percent, from about 10 to about 30 mole percent, from about 10 to about 20 mole percent, from about 20 to about 50 mole percent, from about 20 to about 40 mole percent, from about 20 to about 30 mole percent, from about 30 to about 50 mole percent, from about 30 to about 40 mole percent, from about 15 to about 45 mole percent, from about 25 to about 45 mole percent, from about 25 to about 35, from about 15 to about 35, or from about 20 to about 40 mole percent. In other examples, lactide can be present in about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mole percent, where any of the stated values can form an upper or lower endpoint of a range.

In the disclosed copolymers, MA-FITC can be present in an amount of from about 0.1 to about 2 mole percent. For example, the lactide can be present in an amount of from about 0.1 to about 1.7 mole percent, from about 0.1 to about 1.5 mole percent, from about 0.1 to about 1.3 mole percent, from about 0.1 to about 1.0 mole percent, from about 0.3 to about 1.7 mole percent, from about 0.3 to about 1.5 mole percent, from about 0.3 to about 1.3 mole percent, from about 0.5 to about 1.7 mole percent, from about 0.5 to about 1.5 mole percent, from about 0.5 to about 1.3 mole percent, from about 0.5 to about 1.0, from about 0.7 to about 1.7, or from about 0.5 to about 1.5 mole percent. In other examples, lactide can be present in about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 mole percent, where any of the stated values can form an upper or lower endpoint of a range.

Copolymers containing HMPA, MA-TMA-Cl, and MA-AP, with or without MA-FITC residues, are also contemplated.

In a more general sense, the disclosed copolymers can have the following formula:

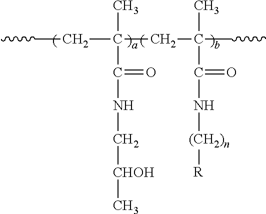

where R is NH₂ or ⁺N(R¹)₃X⁻, wherein each R¹ is, independently of the others a lower alkyl, X is F⁻, Cl⁻, Br⁻, I⁻, SO₄²⁻, CO₂⁻, OH⁻, or CH₃CO₂⁻; n is an integer of from 1 to 6; a and b are integers from 1 to 2000.

Still further disclosed herein are copolymers of N-2-hydroxypropyl methacrylamide (HPMA) and methylacrylamidopropyl quaternary ammonium halide. The quaternary ammonium moiety of the methylacrylamidopropyl quaternary ammonium halide monomer can be

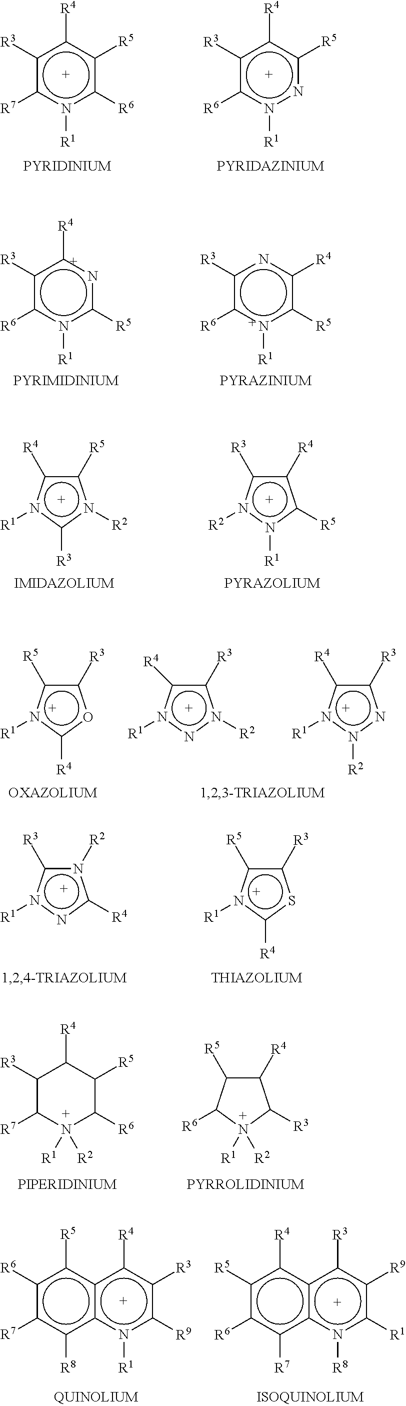

wherein R¹ and R² are independently a C₁-C₆ alkyl group or a C₁-C₆ alkoxyalkyl group, and R³, R⁴, R⁵, R⁶, R⁷, R⁸, and R⁹ (R³-R⁹), when present, are independently H, a C₁-C₆ alkyl, a C₁-C₆ alkoxyalkyl group, or a C₁-C₆ alkoxy group.

### Secondary Components (or Agents)

The disclosed compositions can also comprise one or more secondary components or agents. A secondary component is a bioactive agent, biomolecule, excipient, agent, modifier, surfactant, viscosity modifier, preservative, and/or adjuvant that is added to or admixed with the disclosed polymers. The secondary component can be present during the synthesis of the polymers, but in most instances, the secondary component is added to the polymer composition after the polymer has been synthesized.

In many examples herein, the compositions can have as a secondary component one or more bioactive agents (e.g., pharmaceutical (drug or vaccine), nutrient, biomolecule), contrast agent, imaging agent, dye, targeting moiety, synthetic polymer, magnetic particle, radioopacity agent, and the like. That is, the disclosed compositions can be used as a carrier and delivery device for a wide variety of releasable bioactive agents having curative, therapeutic, or diagnostic value for human or non-human animals. Any of the bioactive agents described herein can be used in this respect. Many of these substances which can be carried by the disclosed compositions are discussed herein.

When the secondary component is a bioactive agent, it can be a drug or other pharmaceutically-active agent use to treat disease or illness. As such, agents including bioactive agents may be used to treat disease or illness in humans or in animals. Any such pharmaceutical can be used as a secondary component. Suitable examples of pharmaceuticals can be found in the Merck Index (13th Edition, Wiley, 2001), The United States Pharmacopeia-National Formulary (USP-NF), and the FDA's Orange book, which are each incorporated by reference herein at least for their teachings of pharmaceuticals. It is also contemplated that potential therapeutic agents including bioactive agents can be suitable secondary components in the disclosed compositions. The resulting pharmaceutical compositions can provide a system for sustained, long-acting continuous delivery of drugs and other biologically-active agents to tissues adjacent to or distant from the application site. In many instances the pharmaceutical compositions are injectable. Classes of disease or illness that may be treated in such a manner include those found in “Goodman & Gilman's The Pharmacological Basis of Therapeutics” (McGraw-Hill, 9th Edition).

Suitable bioactive agents are capable of providing a local or systemic biological, physiological, or therapeutic effect in the biological system to which it is applied. For example, the bioactive agent can act to control infection or inflammation, enhance cell growth and tissue regeneration, control tumor growth, act as an analgesic, and promote anti-cell attachment, among other functions. Other suitable bioactive agents can include anti-viral agents, hormones, antibodies, or therapeutic proteins. Still other bioactive agents include prodrugs, which are agents that are not biologically active when administered but upon administration to a subject are converted to bioactive agents through metabolism or some other mechanism. Additionally, any of the compositions disclosed herein can contain combinations of two or more bioactive agents.

In some examples, the bioactive agents can include substances capable of preventing an infection systemically in the biological system or locally at the defect site, as for example, anti-inflammatory agents such as, but not limited to, pilocarpine, hydrocortisone, prednisolone, cortisone, diclofenac sodium, indomethacin, 6-methyl-prednisolone, corticosterone, dexamethasone, prednisone, and the like; analgesic agents including, but not limited to, salicylic acid, acetaminophen, ibuprofen, naproxen, piroxicam, flurbiprofen, morphine, and the like; local anesthetics including, but not limited to, lidocaine, benzocaine, bupivacaine, levobupivacaine, and the like; immunogens (vaccines) for stimulating antibodies against hepatitis, influenza, measles, rubella, tetanus, polio, rabies, and the like; peptides including, but not limited to, leuprolide acetate (an LH-RH agonist), nafarelin, and the like. Additionally, a substance or metabolic precursor that is capable of promoting growth and survival of cells and tissues or augmenting the functioning of cells is useful, as for example, a nerve growth promoting substance such as a ganglioside, a nerve growth factor, and the like; a hard or soft tissue growth promoting agent such as fibronectin (FN), human growth hormone (HGH), a colony stimulating factor, bone morphogenic protein, platelet-derived growth factor (PDGF), insulin-derived growth factor (IGF-I, IGF-II), transforming growth factor-a (TGF-a), transforming growth factor-13 (TGF-13), epidermal growth factor (EGF), fibroblast growth factor (FGF), interleukin-1 (IL-1), vascular endothelial growth factor (VEGF) and keratinocyte growth factor (KGF), dried bone material, and the like; and antineoplastic agents such as methotrexate, 5-fluorouracil, adriamycin, vinblastine, cisplatin, tumor-specific antibodies conjugated to toxins, tumor necrosis factor, and the like.

Other useful bioactive agents include antibiotics such as acedapsone, acetosulfone sodium, alamecin, alexidine, amdinocillin, amdinocillin pivoxil, amicycline, amifloxacin, amifloxacin mesylate, amikacin, amikacin sulfate, aminosalicylic acid, aminosalicylate sodium, amoxicillin, amphomycin, ampicillin, ampicillin sodium, apalcillin sodium, apramycin, aspartocin, astromicin sulfate, avilamycin, avoparcin, azithromycin, azlocillin, azlocillin sodium, bacampicillin hydrochloride, bacitracin, bacitracin methylene disalicylate, bacitracin zinc, bambermycins, benzoylpas calcium, berythromycin, betamicin sulfate, biapenem, biniramycin, biphenamine hydrochloride, bispyrithione magsulfex, butikacin, butirosin sulfate, capreomycin sulfate, carbadox, carbenicillin disodium, carbenicillin indanyl sodium, carbenicillin phenyl sodium, carbenicillin potassium, carumonam sodium, cefaclor, cefadroxil, cefamandole, cefamandole nafate, cefamandole sodium, cefaparole, cefatrizine, cefazaflur sodium, cefazolin, cefazolin sodium, cefbuperazone, cefdinir, cefepime, cefepime hydrochloride, cefetecol, cefixime, cefmenoxime hydrochloride, cefmetazole, cefmetazole sodium, cefonicid monosodium, cefonicid sodium, cefoperazone sodium, ceforanide, cefotaxime sodium, cefotetan, cefotetan disodium, cefotiam hydrochloride, cefoxitin, cefoxitin sodium, cefpimizole, cefpimizole sodium, cefpiramide, cefpiramide sodium, cefpirome sulfate, cefpodoxime proxetil, cefprozil, cefroxadine, cefsulodin sodium, ceftazidime, ceftibuten, ceftizoxime sodium, ceftriaxone sodium, cefuroxime, cefuroxime axetil, cefuroxime pivoxetil, cefuroxime sodium, cephacetrile sodium, cephalexin, cephalexin hydrochloride, cephaloglycin, cephaloridine, cephalothin sodium, cephapirin sodium, cephradine, cetocycline hydrochloride, cetophenicol, chloramphenicol, chloramphenicol palmitate, chloramphenicol pantothenate complex, chloramphenicol sodium succinate, chlorhexidine phosphanilate, chloroxylenol, chlortetracycline bisulfate, chlortetracycline hydrochloride, cinoxacin, ciprofloxacin, ciprofloxacin hydrochloride, cirolemycin, clarithromycin, clinafloxacin hydrochloride, clindamycin, clindamycin hydrochloride, clindamycin palmitate hydrochloride, clindamycin phosphate, clofazimine, cloxacillin benzathine, cloxacillin sodium, cloxyquin, colistimethate sodium, colistin sulfate, coumermycin, coumermycin sodium, cyclacillin, cycloserine, dalfopristin, dapsone, daptomycin, demeclocycline, demeclocycline hydrochloride, demecycline, denofungin, diaveridine, dicloxacillin, dicloxacillin sodium, dihydrostreptomycin sulfate, dipyrithione, dirithromycin, doxycycline, doxycycline calcium, doxycycline fosfatex, doxycycline hyclate, droxacin sodium, enoxacin, epicillin, epitetracycline hydrochloride, erythromycin, erythromycin acistrate, erythromycin estolate, erythromycin ethylsuccinate, erythromycin gluceptate, erythromycin lactobionate, erythromycin propionate, erythromycin stearate, ethambutol hydrochloride, ethionamide, fleroxacin, floxacillin, fludalanine, flumequine, fosfomycin, fosfomycin tromethamine, fumoxicillin, furazolium chloride, furazolium tartrate, fusidate sodium, fusidic acid, gentamicin sulfate, gloximonam, gramicidin, haloprogin, hetacillin, hetacillin potassium, hexedine, ibafloxacin, imipenem, isoconazole, isepamicin, isoniazid, josamycin, kanamycin sulfate, kitasamycin, levofuraltadone, levopropylcillin potassium, lexithromycin, lincomycin, lincomycin hydrochloride, lomefloxacin, lomefloxacin hydrochloride, lomefloxacin mesylate, loracarbef, mafenide, meclocycline, meclocycline sulfosalicylate, megalomicin potassium phosphate, mequidox, meropenem, methacycline, methacycline hydrochloride, methenamine, methenamine hippurate, methenamine mandelate, methicillin sodium, metioprim, metronidazole hydrochloride, metronidazole phosphate, mezlocillin, mezlocillin sodium, minocycline, minocycline hydrochloride, mirincamycin hydrochloride, monensin, monensin sodiumr, nafcillin sodium, nalidixate sodium, nalidixic acid, natainycin, nebramycin, neomycin palmitate, neomycin sulfate, neomycin undecylenate, netilmicin sulfate, neutramycin, nifuiradene, nifuraldezone, nifuratel, nifuratrone, nifurdazil, nifurimide, nifiupirinol, nifurquinazol, nifurthiazole, nitrocycline, nitrofurantoin, nitromide, norfloxacin, novobiocin sodium, ofloxacin, onnetoprim, oxacillin sodium, oximonam, oximonam sodium, oxolinic acid, oxytetracycline, oxytetracycline calcium, oxytetracycline hydrochloride, paldimycin, parachlorophenol, paulomycin, pefloxacin, pefloxacin mesylate, penamecillin, penicillin G benzathine, penicillin G potassium, penicillin g procaine, penicillin g sodium, penicillin V, penicillin V benzathine, penicillin V hydrabamine, penicillin V potassium, pentizidone sodium, phenyl aminosalicylate, piperacillin sodium, pirbenicillin sodium, piridicillin sodium, pirlimycin hydrochloride, pivampicillin hydrochloride, pivampicillin pamoate, pivampicillin probenate, polymyxin B sulfate, porfiromycin, propikacin, pyrazinamide, pyrithione zinc, quindecamine acetate, quinupristin, racephenicol, ramoplanin, ranimycin, relomycin, repromicin, rifabutin, rifametane, rifamexil, rifamide, rifampin, rifapentine, rifaximin, rolitetracycline, rolitetracycline nitrate, rosaramicin, rosaramicin butyrate, rosaramicin propionate, rosaramicin sodium phosphate, rosaramicin stearate, rosoxacin, roxarsone, roxithromycin, sancycline, sanfetrinem sodium, sarmoxicillin, sarpicillin, scopafungin, sisomicin, sisomicin sulfate, sparfloxacin, spectinomycin hydrochloride, spiramycin, stallimycin hydrochloride, steffimycin, streptomycin sulfate, streptonicozid, sulfabenz, sulfabenzamide, sulfacetamide, sulfacetamide sodium, sulfacytine, sulfadiazine, sulfadiazine sodium, sulfadoxine, sulfalene, sulfamerazine, sulfameter, sulfamethazine, sulfamethizole, sulfamethoxazole, sulfamonomethoxine, sulfamoxole, sulfanilate zinc, sulfanitran, sulfasalazine, sulfasomizole, sulfathiazole, sulfazamet, sulfisoxazole, sulfisoxazole acetyl, sulfisboxazole diolamine, sulfomyxin, sulopenem, sultamricillin, suncillin sodium, talampicillin hydrochloride, teicoplanin, temafloxacin hydrochloride, temocillin, tetracycline, tetracycline hydrochloride, tetracycline phosphate complex, tetroxoprim, thiamphenicol, thiphencillin potassium, ticarcillin cresyl sodium, ticarcillin disodium, ticarcillin monosodium, ticlatone, tiodonium chloride, tobramycin, tobramycin sulfate, tosufloxacin, trimethoprim, trimethoprim sulfate, trisulfapyrimidines, troleandomycin, trospectomycin sulfate, tyrothricin, vancomycin, vancomycin hydrochloride, virginiamycin, and zorbamycin.

Still other useful bioactive agents include hormones such as progesterone, testosterone, and follicle stimulating hormone (FSH) (birth control, fertility-enhancement), insulin, and the like; antihistamines such as diphenhydramine, and the like; cardiovascular agents such as papaverine, streptokinase and the like; anti-ulcer agents such as isopropamide iodide, and the like; bronchodilators such as metaprotemal sulfate, aminophylline, and the like; vasodilators such as theophylline, niacin, minoxidil, and the like; central nervous system agents such as tranquilizer, B-adrenergic blocking agent, dopamine, and the like; antipsychotic agents such as risperidone, narcotic antagonists such as naltrexone, naloxone, buprenorphine; and other like substances. All of these agents are commercially available from suppliers such as Sigma Chemical Co. (Milwaukee, Wis.).

Included among bioactive agents that are suitable for incorporation into the disclosed compositions are therapeutic drugs, e.g., anti-inflammatory agents, anti-pyretic agents, steroidal and non-steroidal drugs for anti-inflammatory use, hormones, growth factors, contraceptive agents, antivirals, antibacterials, antibiotics, antifungals, analgesics, hypnotics, sedatives, tranquilizers, anti-convulsants, muscle relaxants, local anesthetics, anesthetics, antispasmodics, antiulcer drugs, peptidic agonists, sympathiomimetic agents, cardiovascular agents, antitumor agents, oligonucleotides and their analogues and so forth. The bioactive agent is added in pharmaceutically active amounts.

Further non-limiting examples of bioactive agents include, small molecule, a peptide, a protein, an enzyme (e.g., a kinase, a phosphatase, a methylating agent, a factor, a protease, a transcriptase, an endonuclease, a ligase, and the like), a vaccine, an antibody and/or fragment thereof, a nucleic acid (e.g., an oligonucleotide, a prime, a probe, an aptamer, a ribozyme, etc.), a lipid, a carbohydrate, a steroid, a hormone, a vitamin. In certain aspects, the bioactive agent can be a biomolecule (which are likely bioactive as well). Examples of biomolecules also include, but are not limited to, a small molecule, a peptide, a protein, an enzyme (e.g., a kinase, a phosphatase, a methylating agent, a factor, a protease, a transcriptase, an endonuclease, a ligase, and the like), a vaccine, an antibody and/or fragment thereof, a nucleic acid (e.g., an oligonucleotide, a prime, a probe, an aptamer, a ribozyme, etc.), a lipid, a carbohydrate, a steroid, a hormone, a vitamin. “Small molecule” as used herein, is meant to refer to a composition, which has a molecular weight of less than about 5 kD, for example, less than about 4 kD. Small molecules can be nucleic acids (e.g., DNA, RNA), peptides, polypeptides, peptidomimetics, carbohydrates, lipids, factors, cofactors, hormones, vitamins, steroids, trace elements, or other organic (carbon containing) or inorganic molecules. Such biomolecules can be obtained commercially or can be synthesized or isolated from natural sources by methods known in the art.

There are a variety of compositions disclosed herein where the secondary component (e.g., biomolecule) can comprise an amino acid based molecule, including for example peptides, proteins, enzymes, vaccines, and antibodies. Thus, as used herein, “amino acid,” means the typically encountered twenty amino acids which make up polypeptides. Non-limiting examples of peptides include native peptides, synthetic peptides, biologically active peptides, factors, growth factors, and so on including, but not limited to, bioactive peptides and classes of bioactive peptides described in the “Handbook of Biologically Active Peptides” (A. J. Krastin, Editor; Academic Press, 2006).

In addition, it further includes less typical constituents which are both naturally occurring, such as, but not limited to formylmethionine and selenocysteine, analogs of typically found amino acids, and mimetics of amino acids or amino acid functionalities. Non-limiting examples of these and other molecules are discussed herein.

As used herein, the terms “peptide” and “protein” refer to a class of compounds composed of amino acids chemically bound together. Non-limiting examples of these and other molecules are discussed herein. In general, the amino acids are chemically bound together via amide linkages (CONH); however, the amino acids can be bound together by other chemical bonds known in the art. For example, the amino acids can be bound by amine linkages. “Peptide” as used herein includes oligomers of amino acids and small and large peptides, including naturally occurring or engineered polypeptides and proteins. It is understood that the terms “peptide” and “protein” can be used interchangeably herein.

Methods for producing such peptides and proteins are well known. One method of producing the disclosed proteins is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof (Grant, Synthetic Peptides: A User Guide. W.H. Freeman and Co., N.Y. 1992; Bodansky and Trost, Ed. Principles of Peptide Synthesis. Springer-Verlag Inc., N.Y., 1993, which are incorporated by reference herein at least for their teachings of peptide synthesis.)

In another example, the secondary component can comprise an antibody or fragment thereof. Antibodies or fragments thereof can be considered biomolecules, imaging agents, and/or target moieties, as the terms are used herein. The term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain has a variable 20 domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one 25 of two clearly distinct types, called kappa and lambda, based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse.

The heavy chain constant domains that correspond to the different classes of immunoglobulins are called alpha, delta, epsilon, gamma, and mu, respectively. The term “antibody” as used herein is meant to include intact molecules as well as fragments thereof, such as, for example, Fab and F(ab′)₂, which are capable of binding the epitopic determinant. The term “antibody” also includes monoclonal and polyclonal antibodies, anti-idiopathic, and humanized antibodies.

As used herein, the term “antibody or fragments thereof” encompasses chimeric antibodies and hybrid antibodies, with dual or multiple antigen or epitope specificities, and fragments, such as F(ab′)2, Fab′, Fab and the like, including hybrid fragments. Such antibodies and fragments can be made by techniques known in the art (see Harlow and Lane. Antibodies, A Laboratory Manual. Cold Spring Harbor Publications, N.Y., 1988). Such antibodies and fragments thereof can be screened for specificity and activity according to the methods disclosed herein.

Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference for at least its teaching of antibody conjugates. The fragments, whether attached to other sequences or not, include insertions, deletions, substitutions, or other selected modifications of particular regions or specific amino acid residues. Methods of producing and/or isolating antibodies as disclosed herein are well known.

There are also a variety of compositions disclosed herein where the secondary component can comprise a nucleic acid based molecule. Thus, as used herein, “nucleic acid” means a molecule made up of, for example, nucleotides, nucleotide analogs, or nucleotide substitutes. Non-limiting examples of these and other molecules are discussed herein. A nucleic acid can be double stranded or single stranded. Nucleic acid is also meant to include oligonucleotides, siRNA, DNA, plasmid, and the like.

As used herein, “nucleotide” is a molecule that contains a base moiety, a sugar moiety and a phosphate moiety. Nucleotides can be linked together through their phosphate moieties and sugar moieties creating an internucleoside linkage. The base moiety of a nucleotide can be adenine-9-yl (A), cytosine-1-yl (C), guanine-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

“Nucleotide analog,” as used herein, is a nucleotide which contains some type of modification to the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

“Nucleotide substitutes,” as used herein, are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Included herein are nucleic acid complexes or nucleic acid conjugates. It is also possible to link other types of molecules to nucleotides or nucleotide analogs to make conjugates or complexes that can enhance, for example, cellular uptake and cellular transfection. Conjugates or complexes can be chemically linked to the nucleotide or nucleotide analogs. Such conjugates include but are not limited to lipid moieties such as a cholesterol moiety (Letsinger et al., Proc Natl Acad Sci USA, 86:6553-6, 1989, which is incorporated by reference herein at least for its teachings of nucleic acid conjugates). Moreover, conjugates or complexes may be non-covalently associated by ionic or charge-charge or hydrophobic or by van der Waal's or by other non-covalent means. Examples of nucleic acid complexes or conjugates include nucleic acid polymer conjugates such as polyplexes in which the nucleic acid (such as a plasmid or DNA or siRNA) is covalently or non-covalently associated with a polymer. As used herein, the term nucleic acid includes such conjugates, complexes, analogs, polyplexes, and variants of nucleic acids.

Nucleic acids, such as those described herein, can be made using standard chemical synthetic methods or can be produced using enzymatic methods or any other known method. Such methods can range from standard enzymatic digestion followed by nucleotide fragment isolation (see for example, Sambrook et al., Molecular Cloning: A Laboratory Manual, 3d Edition, Cold Spring Harbor Laboratory Press, Cold Spring Harbor, N.Y., 2001, Chapters 5, 6) to purely synthetic methods, for example, by the cyanoethyl phosphoramidite method using a Milligen or Beckman System Plus DNA synthesizer (for example, Model 8700 automated synthesizer of Milligen-Biosearch, Burlington, Mass. or ABI 30 Model 380B). Synthetic methods useful for making oligonucleotides are also described by Ikuta et al., Ann Rev Biochem 53:323-56, 1984, (phosphotriester and phosphite-triester methods), and Narang et al., Methods Enzymol 65:610-20, 1980, (phosphotriester method). Protein nucleic acid molecules can be made using known methods such as those described by Nielsen et al., Bioconjug Chem, 5:3-7, 1994. (Each of these references is incorporated by reference herein at least for their teachings of nucleic acid synthesis.)

Also, the secondary component can comprise an imaging agent, which is a chemical compound that can produce a detectable signal, either directly or indirectly. Many such imaging agents are known to those of skill in the art. Examples of imaging agents suitable for use in the disclosed compositions and method are radioactive isotopes, fluorescent molecules, magnetic particles (including nanoparticles), metal particles (including nanoparticles), phosphorescent molecules, enzymes, antibodies, and ligands. Imaging agents that combine two or more of the moieties disclosed herein are also considered imaging moieties.

Any of the known imaging agents can be used with the disclosed compositions. Methods for detecting and measuring signals generated by imaging agents are also known to those of skill in the art. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by detection or visualization of the product of a reaction catalyzed by the enzyme; antibodies can be detected by detecting a secondary detection label coupled to the antibody.

In one example, the disclosed imaging agents can comprise a fluorescent imaging agent. A fluorescent imaging agent is any chemical moiety that has a detectable fluorescence signal. This imaging agent can be used alone or in combination with other imaging agents. Examples of suitable fluorescent agents that can be used in the compositions and methods disclosed herein include, but are not limited to, fluorescein (FITC), 5-carboxyfluorescein-N-hydroxysuccinimide ester, 5,6-carboxymethyl fluorescein, nitrobenz-2-oxa-1,3-diazol-4-yl (NBD), fluorescamine, OPA, NDA, indocyanine green dye, the cyanine dyes (e.g., Cy3, Cy3.5, Cy5, Cy5.5 and Cy7), 4-acetamido-4′-isothiocyanatostilbene-2,2′ disulfonic acid, acridine, acridine isothiocyanate, 5-(2′-aminoethypaminonaphthalene-1-sulfonic acid (EDANS), 4-amino-N43-vinylsulfonyl)phenylinaphthalimide-3,5 disulfonate, N-(4-anilino-1-naphthyl)maleimide, anthranilamide, BODIPY, Brilliant Yellow, coumarin, 7-amino-4-methylcoumarin (AMC, Coumarin 120), 7-amino-4-trifluoromethylcoumarin (Coumaran 151), cyanosine, 4′,6-diaminidino-2-phenylindole (DAPI), 5′,5″-dibromopyrogallol-sulfonaphthalein (Bromopyrogallol Red), 7-diethylamino-3-(4′-isothiocyanatophenyl)-4-methylcoumarin diethylenetriamine pentaacetate, 4,4′-diisothiocyanatodihydro-stilbene-2,2′-disulfonic acid, 4,4′-diisothiocyanatostilbene-2,2′-disulfonic acid, 5-[dimethylamino]naphthalene-lsulfonyl chloride (DNS, dansylchloride), 4-(4′-dimethylaminophenylazo)benzoic acid (DABCYL), 4-dimethylaminophenylazophenyl-4′-isothiocyanate (DABITC), eosin, eosin isothiocyanate, erythrosin B, erythrosine, isothiocyanate, ethidium bromide, ethidium, 5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein isothiocyanate, IR144, IR1446, Malachite Green isothiocyanate, 4-methylumbelliferone, ortho cresolphthalein, nitrotyrosine, pararosaniline, Phenol Red, B-phycoerythrin, o-phthaldialdehyde, pyrene, pyrene butyrate, succinimidyl 1-pyrene butyrate, Reactive Red 4 (Cibacron[R] Brilliant Red 3B-A), 6-carboxy-X-rhodamine (ROX), 6-carboxyrhodamine (R6G), lissamine rhodamine B sulfonyl chloride rhodamine (Rhod), 5,6-tetramethyl rhodamine, rhodamine B, rhodamine 123, rhodamine X isothiocyanate, sulforhodamine B, sulforhodamine 101, sulfonyl chloride derivative of sulforhodamine 101 (Texas Red), N,N,N′,N′-tetramethyl-6-carboxyrhodamine (TAMRA), tetramethyl rhodamine, tetramethyl rhodamine isothiocyanate (TRITC), riboflavin, rosolic acid, coumarin-6, and the like, including combinations thereof. These fluorescent imaging moieties can be obtained from a variety of commercial sources, including Molecular Probes, Eugene, Oreg. and Research Organics, Cleveland, Ohio, or can be synthesized by those of ordinary skill in the art.

In another example, the disclosed imaging agents can comprise a Magnetic Resonance Imaging (MRI) agent. A MRI agent is any chemical moiety that has a detectable magnetic resonance signal or that can influence (e.g., increase or shift) the magnetic resonance signal of another agent. This type of imaging agent can be used alone or in combination with other imaging agent. In still another example, a gadolinium-based MRI agent can serve as an imaging agent. An example of a suitable MRI agent that can be incorporated into the disclosed imaging agents is para-amino-benzyl diethylenetriaminepentaacetic acid (p-NH₂-Bz-DTPA, Compound 7), a conjugable form of diethylenetriaminepentaacetic acid (DTPA), which is known to strongly bind gadolinium and is approved for clinical use as a magnetic resonance contrast agent. Others have successfully bound similar MRI contrast agents to PAMAM™ (Kobayashi et al., Bioconjugate Chem 12:100-107, 2001; Kobayashi et al., Mag Res in Medicine 46:579-85, 2001) dendrimers for in vivo small animal imaging; these references are incorporated by reference herein at least for their teachings of MRI agents. Incorporation of an MRI agent on a large macromolecule such as a dendrimeric substrate as disclosed herein can allow large T1 relaxation (high contrast) and multiple copies of agent on a single molecule, which can increase signal. By combining an MRI imaging agent and, for example, a fluorescent imaging agent, the resulting agent can be detected, imaged, and followed in real-time via MR I.

Other imaging agents include PET agents that can be prepared by incorporating an 18F or a chelator for 64Cu or 68Ga. Also, addition of a radionuclide can be used to facilitate SPECT imaging or delivery of a radiation dose.

Plasticizers

Plasticizers should be biocompatible with and soluble in the disclosed compositions. Suitable plasticizers can be solvents, lipids, oils, fatty acids, surfactants, solubilizers, and polymeric additives. Examples of biocompatible solvents include fatty acids, oils, aromatic alcohols, lower alkyl esters of aryl acids, lower aralkyl esters of aryl acids, aryl ketones, aralkyl ketones, lower alkyl ketones, and lower alkyl esters of citric acid; benzoic acid derivatives; phthalic acid derivatives; and combinations thereof. Suitable examples of plasticizers include, but are not limited to, lactic acid, glycolic acid, hydroxybutyric acid, caprolactone, ethyl caproate, ethyl glycolate, ethyl oleate, benzyl benzoate, ethyl benzoate, lauryl lactate, benzyl alcohol, lauryl alcohol, glycofurol, ethyl acetate, ethanol, butanol, isopropyl alcohol, propanol, tocopherol, polyethylene glycol, triacetin, a triglyceride, an alkyltriglyceride, a diglyceride, rapeseed oil, sesame oil, peanut oil, castor oil, olive oil, cottonseed oil, perfluorocarbon, N-methylpyrrolidone, N-methyl-2-pyrrolidione, DMSO, glycerol, oleic acid, glycofurol, lauryl lactate, perfluorocarbon, propylene carbonate methyl benzoate, ethyl benzoate, n-propyl benzoate, isopropyl benzoate, butyl benzoate, isobutyl benzoate, sec-butyl benzoate, tert-butyl benzoate, isoamyl benzoate, benzyl benzoate, triethyl citrate, tributyl citrate, and combinations or mixtures thereof. Other examples include, but are not limited to, caprolactone, ethyl caproate, benzyl alcohol, ethyl acetate, acetone, butanone, methyl alcohol, butyl alcohol, methylene chloride, DMF, and the like, including mixtures thereof. Additional examples of plasticizers include, but are not limited to, glycerol triacetate, acetylated monoglycerides, citric acid esters, triethyl citrate, triethyl acetyl citrate, tributyl citrate, tributyl acetyl citrate, dibutyl phthalate, dibutyl sebacate, diethyloxalate, diethylmalate, diethylfumarate, diethylsuccinate, diethylmalonate, diethyltartrate, phthalic acid esters, diethylphthalate, dimethylphthalate, glycerin, glycerol, glyceryl triacetate, glyceryltributyrate, mineral oil and lanolin alcohols, petrolatum and lanolin alcohols, polyethylene glycols, propylene glycols, copolymers of polypropylene glycol and polyethylene glycol (including poloxamers), polyvinyl pyrrolidone, polysorbate 80, and the like, including mixtures thereof.

Surfactants

The disclosed compositions can also comprise agents such as surfactants. A “surfactant” as used herein is a molecule composed of hydrophilic and hydrophobic groups (i.e., an amphiphile). The surfactant can be an ionic or nonionic surfactant. For example, the disclosed compositions can comprise an anionic surfactant. Any anionic surfactants can be used. Suitable anionic surfactants are commonly used in detergents, shampoos, soaps, etc., and can be obtained commercially or prepared by methods known in the art. They include, but are not limited to, alkylbenzene sulfonates (detergent), fatty acid based surfactants, lauryl sulfate (e.g., a foaming agent), di-alkyl sulfosuccinate (e.g., a wetting agent), lignosulfonates (e.g., a dispersant), and the like, including mixtures thereof. In other examples, linear alkylbenzene sulphonic acid, sodium lauryl ether sulphate, alpha olefin sulphonates, phosphate esters, sodium sulphosuccinates, hydrotropes, and the like, including mixtures thereof, can be used.

In other examples, the disclosed compositions can comprise a cationic surfactant. Any cationic surfactant can be used. Suitable cationic surfactants included, but are not limited to, quaternary ammonium compounds, imidazolines, betaines, etc. Such cationic surfactants can be obtained commercially or can be prepared by methods known in the art. In still other examples, the disclosed compositions can comprise a nonionic surfactant. Any nonionic surfactant can be used. Suitable nonionic surfactants do not ionize in aqueous solution, because their hydrophilic group is of a non-dissociable type, such as alcohol, phenol, ether, ester, or amide. They can be classified as ethers (e.g., polyhydric alcohols such as glycerin, solbitole, sucrose, etc.), fatty acid esters (e.g., glycerin fatty acid ester, sobitan fatty acid ester, sucrose fatty acid ester, etc.), esters (e.g., compounds made by applying, for example, ethylene oxide to a material having hydroxyl radicals such as high alcohol, alkyl-phenol, and the like), ether/esters (e.g., compounds made by applying, for example, the ethylene oxide to the fatty acid or polyhydric alcohol fatty acid ester, having both ester bond and ether bond in the molecule), and other types (e.g., the fatty acid alkanol-amide type or the alkylpolyglyceride type). A particularly suitable nonionic surfactant is poly(vinyl alcohol). Other suitable examples of nonionic surfactants can include, but are not limited to, alcohol ethoxylates and alkyl phenol ethyoxylates, fatty amine oxides, alkanolamides, ethylene oxide/propylene oxide block copolymers, alkyl amine ethoxylates, tigercol lubricants, and the like, including mixtures thereof.

In yet other examples, the disclosed compositions can comprise dipolar surfactants. Any dipolar surfactant can be used. Suitable dipolar surfactants (called amphoteric or zwitterionic) exhibit both anionic and cationic dissociation. Suitable examples of dipolar surfactants include, but are not limited to, products like betaines or sulfobetaines and natural substances such as amino acids and phospholipids. In one example, the betaines disclosed in U.S. Pat. Nos. 6,852,816; 6,846,795; 6,846,352; and 6,849,426, which are incorporated by reference in their entireties, can be used herein.

Other examples of suitable surfactants include natural surfactants, which can have their source from plant or animal organs. In another example, a boloform surfactant can be used. A boloform surfactant is a surfactant that has two hydrophilic head groups at opposite ends of a hydrophobic tail. Mixtures of these surfactants can also be used in the compositions and methods disclosed herein.

Additives

Compositions comprising the disclosed polymers can also comprise other agents including additives or excipients. For example, the disclosed compositions can contain pH buffers, organic acids (e.g., formic, acetic, propionic, benzoic, maleic, oxalic acids, and the like), mineral acids (e.g., HCl, HBr, H2SO₄, H₃PO₄, and the like), bases (e.g., NaOH, KOH, Et₃N, Na₂CO₃, NaHCO₃, KHCO₃, and the like), preservatives, dyes, antioxidants (e.g., ascorbic acid and tocopherols), wetting, emulsifying, suspending agents, flocculating, and dispensing agents.

The disclosed compositions can also contain other additives for preventing the action of microorganisms. This can be accomplished by various antimicrobial and/or antifungal agents, for example, parabens, chlorobutanol, phenol, sorbic acid, quaternary ammonium compounds, and the like.

It may also be desirable to include binders such as carboxymethylcellulose, alignates, gelatin, polyvinyl pyrrolidone, sucrose, and acacia, humectants such as glycerol, wetting agents such as cetyl alcohol and glycerol monostearate, adsorbents such as kaolin and bentonite, and lubricants such as talc, calcium stearate, magnesium stearate, polyethylene glycols, polypropylene glycols, copolymers of polyethylene glycol and polypropylene glycol, sodium lauryl sulfate, or mixtures thereof.

Suitable flocculating agents that can be used include, but are not limited to, aluminum salts (e.g., aluminium sulphate), ferrous salts, and ferric salts (e.g., ferric sulphate and ferric chloride). Suitable suspending agents can include, for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like. The disclosed compositions can also comprise solubilizing agents and emulsifiers, as for example, ethyl alcohol, isopropyl alcohol, ethyl carbonate, ethyl acetate, benzyl alcohol, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like.

Pharmaceutical Formulations

Also, pharmaceutical formulations comprising the disclosed polymers and one or more bioactive agents are disclosed herein. A suitable pharmaceutical formulation can comprise any of the disclosed polymers and bioactive agents, along with a pharmaceutically acceptable carrier. In many examples, the polymers disclosed herein are themselves pharmaceutically acceptable carriers. The pharmaceutical formulations disclosed herein can be used therapeutically or prophylactically.

By “pharmaceutically acceptable” is meant a material that is not biologically or otherwise undesirable, i.e., the material may be administered to a subject without causing any undesirable biological effects or interacting in a deleterious manner with any of the other components of the pharmaceutical formulation in which it is contained. The carrier would naturally be selected to minimize any degradation of the active ingredient and to minimize any adverse side effects in the subject, as would be well known to one of skill in the art.

Pharmaceutical carriers are known to those skilled in the art. These most typically would be standard carriers for administration of drugs to humans, including solutions such as sterile water, saline, and buffered solutions at physiological pH. Suitable carriers and their formulations are described in Remington: The Science and Practice of Pharmacy, 21St Ed., Lippincott Williams & Wilkins, Philadelphia, Pa., 2005, which is incorporated by reference herein for its teachings of carriers and pharmaceutical formulations. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution can be from about 5 to about 8 (e.g., from about 7 to about 7.5). Further carriers include sustained release preparations such as semipermeable matrices of solid hydrophobic polymers containing the disclosed compounds, which matrices are in the form of shaped articles, e.g., films, liposomes, microparticles, or microcapsules. It will be apparent to those persons skilled in the art that certain carriers can be more preferable depending upon, for instance, the route of administration and concentration of composition being administered. Other compounds can be administered according to standard procedures used by those skilled in the art.

Pharmaceutical formulations can include additional carriers, as well as thickeners, diluents, buffers, preservatives, surface active agents and the like in addition to the compounds disclosed herein. Pharmaceutical formulations can also include one or more additional active ingredients such as antimicrobial agents, anti-inflammatory agents, anesthetics, and the like. The pharmaceutical formulation can be administered in a number of ways depending on whether local or systemic treatment is desired, and on the area to be treated.

Administration can be topically (including ophthalmically, vaginally, rectally, intranasally), orally, by inhalation, or parenterally, for example by intravenous drip, subcutaneous, intraperitoneal or intramuscular injection. The disclosed compounds can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, marine oils, and injectable organic esters such as ethyl oleate. Aqueous carriers include water, alcoholic/aqueous solutions, and emulsions or suspensions, including saline and buffered media. Parenteral vehicles include sodium chloride solution, Ringer's dextrose, dextrose and sodium chloride, lactated Ringer's, and fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical formulations for topical administration may include ointments, lotions, creams, gels, drops, suppositories, sprays, liquids and powders. Conventional pharmaceutical carriers, aqueous, powder or oily bases, thickeners and the like can be desirable.

Pharmaceutical formulations for oral administration include, but are not limited to, powders or granules, suspensions or solutions in water or non-aqueous media, capsules, gel-caps, sachets, or tablets. Thickeners, flavorings, diluents, emulsifiers, dispersing aids, or binders can be desirable.

Some of the formulations can potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, pamoic acid and fumaric acid, or by reaction with an inorganic base such as sodium hydroxide, ammonium hydroxide, potassium hydroxide, and organic bases such as mono-, di-, trialkyl and aryl amines and substituted ethanolamines.

Pharmaceutical Kits

Also disclosed are kits or packages of pharmaceutical formulations designed for use in the regimens described herein. These kits can be designed for daily oral delivery over 4-hour, 6-hour, 8-hour, 12-hour, 24-hour, 48-hour, 72-hour, 7-day, 10-day, 21-day, or 30-day cycle, among others, and also for one oral delivery per day. When the compositions are to be delivered continuously, a package or kit can include the composition in each tablet. When the compositions are to be delivered with periodic discontinuation, a package or kit can include placebos on those days when the composition is not delivered.

The kits can also be organized to indicate a single oral formulation or combination of oral formulations to be taken on each day of the cycle, including oral tablets to be taken on each of the days specified, for example, one oral tablet will contain each of the combined daily dosages indicated.

In one example, a kit can include a single phase of a daily dosage of the disclosed 30 compounds over a 4-hour, 6-hour, 8-hour, 12-hour, 24-hour, 48-hour, 72-hour, 7-day, 10-day, 21-day, or 30-day cycle.

### Methods

The disclosed compositions have uses in therapy for cardiopulmonary diseases, among others. While not wishing to be bound by theory, the disclosed HPMA polymers can interact with the negatively charged endothelial glycocalyx without being sequestered inside endothelial cells. The copolymers containing quaternary amine functions tend to be less reactive than primary amines in cellular systems. One explanation for this observation is that ammonium is less chemically labile than is a primary amine and therefore is less likely to react with receptors involved in cellular uptake. The copolymers disclosed herein were synthesized to contain increasing molar ratios of primary- and quaternary amine conjugates in order to maximize binding to the glycocalyx while minimizing cell uptake.

The safety of the disclosed HPMA copolymers is demonstrated by noting the reduction of LDH release in the presence of quaternary ammonium functional groups as opposed to copolymers containing primary amine side chains. Further, the question of the disclosed copolymer binding to the glycocalyx is addressed by incubating fluorescently labeled copolymers with endothelial monolayers and showing their continued presence after washing steps. The efficacy of the disclosed copolymers is demonstrated in their ability to attenuate both albumin diffusion and hydraulic conductivity across endothelial monolayers.

Disclosed herein are methods of treating a patient diagnosed with a cardiopulmonary disease comprising contacting lung microvasculature of the patient with an effective amount of a copolymer as disclosed herein, e.g., P-TMA-Cl and P-AP. Further polymers that can be used are copolymers of HPMA and methylacrylamidopropyl quaternary ammonium halide. The cardiopulmonary disease can be pulmonary edema, such as that caused by acute lung injury or acute respiratory distress syndrome. Such acute respiratory distress is often associated with burn victims due to smoke inhalation. Other disease states that can be treated are those associated with cardiopulmonary bypass surgery, solid organ transplant, and major vascular surgery. Another example of a disease treatable by the compositions disclosed herein is Adult Respiratory Distress Syndrome (ARDS).

Dosage

When used in the above described methods or other treatments, or in the pharmaceutical formulations (e.g., a composition as disclosed herein with a bioactive agent) disclosed herein, an “effective amount” of one of the disclosed bioactive agents can be employed in pure form or, where such forms exist, in pharmaceutically acceptable salt form, and with or without a pharmaceutically acceptable excipient, carrier, or other additive.

The specific effective amount for any particular subject will depend upon a variety of factors including the disorder being treated and the severity of the disorder; the identity and activity of the specific composition employed; the age, body weight, general health, sex and diet of the patient; the time of administration; the route of administration; the rate of excretion of the specific composition employed; the duration of the treatment; drugs used in combination or coincidental with the specific composition employed and like factors well known in the medical arts. For example, it is well within the skill of the art to start doses of a composition at levels lower than those required to achieve the desired therapeutic effect and to gradually increase the dosage until the desired effect is achieved. One can also evaluate the particular aspects of the medical history, signs, symptoms, and objective laboratory tests that are known to be useful in evaluating the status of a subject in need of attention for the treatment of ischemia-reperfusion injury, trauma, drug/toxicant induced injury, neurodegenerative disease, cancer, or other diseases and/or conditions. These signs, symptoms, and objective laboratory tests will vary, depending upon the particular disease or condition being treated or prevented, as will be known to any clinician who treats such patients or a researcher conducting experimentation in this field. For example, if based on a comparison with an appropriate control group and/or knowledge of the normal progression of the disease in the general population or the particular individual: 1) a subject's physical condition is shown to be improved (e.g., a tumor has partially or fully regressed), 2) the progression of the disease or condition is shown to be stabilized, or slowed, or reversed, or 3) the need for other medications for treating the disease or condition is lessened or obviated, then a particular treatment regimen will be considered efficacious. If desired, the effective daily dose can be divided into multiple doses for purposes of administration. Consequently, single dose compositions can contain such amounts or submultiples thereof to make up the daily dose.

In a further aspect, an effective amount can be determined by preparing a series of compositions comprising varying amounts of bioactive agents and determining the release characteristics in vivo and in vitro and matching these characteristics with specific pharmaceutical delivery needs, inter alia, subject body weight, disease condition and the like. The dosage can be adjusted by the individual physician or the subject in the event of any counterindications. Dosage can vary, and can be administered in one or more dose administrations daily, for one or several days. Guidance can be found in the literature for appropriate dosages for given classes of pharmaceutical products.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, pH, etc.) but some errors and deviations should be accounted for. Unless indicated otherwise, parts are parts by weight, temperature is in ° C. or is at ambient temperature, and pressure is at or near atmospheric. There are numerous variations and combinations of conditions, e.g., component concentrations, temperatures, pressures, and other reaction ranges and conditions that can be used to optimize the product purity and yield obtained from the described process. Only reasonable and routine experimentation will be required to optimize such process conditions.

Data are expressed as means±SD with differences between groups assessed using nonparametric Dunn's test to show differences between 3 or more groups with unequal variances or paired t-tests when evaluating data between pressure increases. Graphics were generated in Kaleidograph (Synergy Software: Reading, Pa.) and significance is concluded if P<0.05.

Certain materials, compounds, compositions, and components disclosed herein can be obtained commercially or readily synthesized using techniques generally known to those of skill in the art. For example, the starting materials and reagents used in preparing the disclosed compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplements (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

### Example 1

### Copolymer Synthesis/Characterization

Copolymer of HPMA and either MA-TMA-Cl or MA-AP were synthesized in varying feed ratios. The monomers were obtained from Polysciences (Warrington, Pa.). The copolymers were synthesized by radical polymerization of HPMA with either MA-TMA-Cl or MA-AP monomers using methanol as the solvent with AIBN (2,2-azabisisobutryonitrile) as initiator. The comonomers concentration in polymerization mixture was 12.5 wt % and the AIBN concentration was 0.6 wt %. For fluorescence studies, 1 mol % MA-FITC was added to polymerization mixture. Radical polymerization was allowed to proceed under nitrogen atmosphere at 55° C. for 24 h. Molecular weight and molecular distribution were determined using AKTA FPLC (GE Healthcare) equipped with UV and RI detectors using a Superose 6 column in PBS pH 7.4. Quaternary amine content was determined by titration of chlorine counterion using AgNO₃ and primary amine content was determined by ninhydrin assay (Samejima et al., Anal Biochem 42(1)237-47, 1971).

TABLE 1
Copolymer characterization data

| | | | Feed | Polymer | Molecular |
|---|---|---|---|---|---|
| | | | composition | composition | weight (kDa)^{c} |
| No. | Name | Structure of side chain | (mol %) | (mol %) | (PDI) |
| 1 | 40P-AP | —NH(CH₂)₃—NH₂ | 40 | 35^{a} | ND |
| 2 | 80P-AP | —NH(CH₂)₃—NH₂ | 80 | 70^{a} | ND |
| 3 | 40P-TMA Cl | —NH(CH₂)₃—N(CH₃)₃⁺Cr⁻ | 40 | 46^{b} | 53.8 (1.8) |
| 4 | 80P-TMA Cl | —NH(CH₂)₃—N(CH₃)₃⁺Cr⁻ | 80 | 70^{b} | 65.6 (2.1) |

ND: not determined. Fluorescent copolymers were polymerized with 1 mol % MA-FITC in feed composition and content of FITC was determined by UV spectroscopy using a molar extinction
coefficient of 80,000 M⁻¹cm⁻¹.
^{a}Determined by ninhydrin test.
^{b}Determined by titration with AgNo3.
^{c}Determined by size exclusion chromatography.

Copolymer structures are provided below:

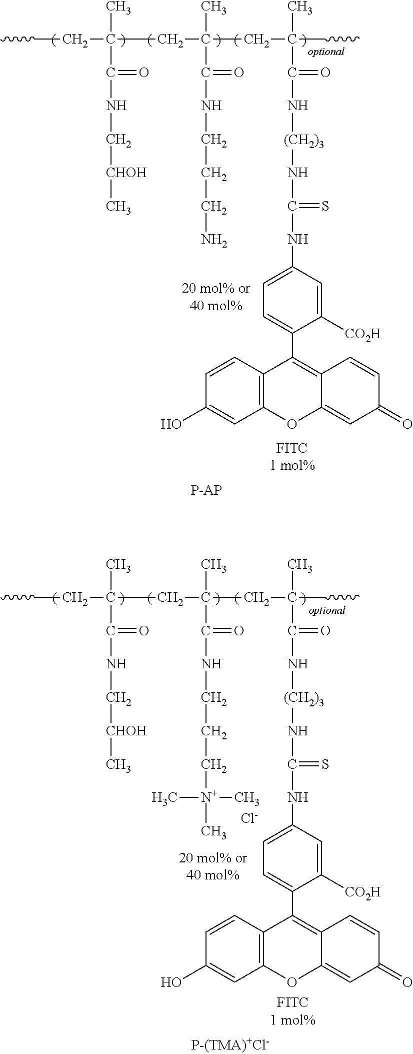

### Example 2

### Cytotoxicity

Release of lactate dehydrogenase (LDH) was evaluated using a commercially available bioassay kit (BioVision, Moutain View, Calif.). LDH release is released from cells prior to apoptosis and is therefore a standard marker for cell death.

Cell suspension (1.5×10⁴ cells/well) was added to a 96-well plate and incubated at 37° C. for 30 min with increasing concentrations of 40P-TMA-Cl or 40P-AP. Post-incubation, cells were centrifuged at 250×g and the supernatant was added to reaction solution provided in kit. After reacting at room temperature for 30 min, absorbance was evaluated on a GENious 96-well plate reader (Tecan, Durham, N.C.) at 492 nm. Data are presented as:

Cytotoxicity (%)=((sample−C_{L})/(C_{H}−C_{L}))×100

where C_{L} represents low control cells receiving no treatment and C_{H} represents high control cells receiving 1% Triton X-100. Statistics were calculated using N=3 replicates per group.

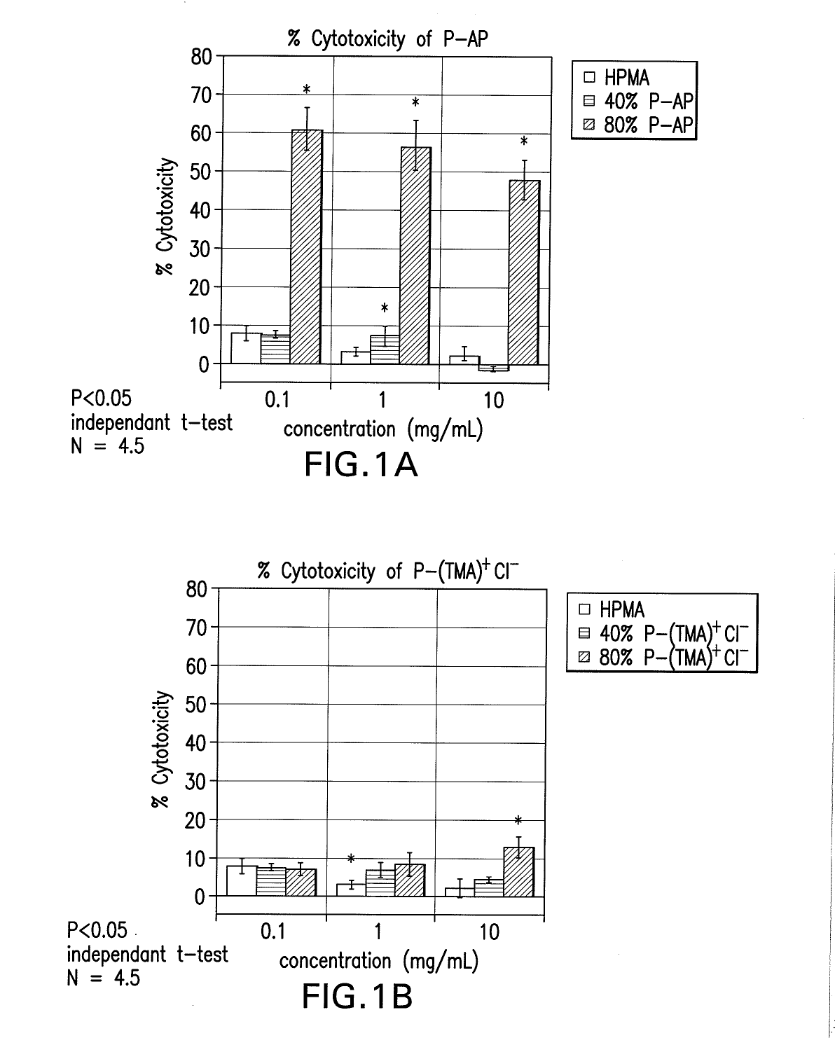

LDH release was minimal in groups treated with P-HPMA or P-TMA-Cl; only at the highest concentration of polymer and highest mol % of TMA (80P-TMA-Cl at 10 mg/mL) does the cytotoxicity exceed 10% FIG. 1B. At the same concentration and composition percent, primary amine comonomers elicit 5-6 fold increases in LDH release (FIG. 1A). Marked cytotoxicity occurs in the presence of copolymers containing a high percentage of primary amines, but at 40 mol % both cationic polymers behave similarly. Copolymers possessing quaternary amines exhibit significantly less cytotoxicity than primary amine copolymers at higher feed compositions.

### Example 3

### Copolymer-Cell Interactions

Bovine lung endothelial (BLMVEC) monolayers were subcultured on glass coverslips pretreated with 0.4% gelatin (1 h) and 100 μg/mL fibronectin (1 hour) and cultured to confluency. Cells were rinsed with PBS (pH 7.4) then incubated 30 min with 40P-(FITC)-TMA-Cl copolymer at a concentration of 1 mg/mL in phenol red-free DMEM supplemented with 1% BSA, 25 mM HEPES at pH 7.4. Cells were subsequently washed in PBS and fixed for 10 min in 2% paraformaldehyde at room temperature. Monolayers were rinsed in PBS then incubated at room temperature with 0.1% Triton X-100 to permeablize cell membranes. Phalloidin was dissolved in methanol (6.6 μM), diluted to 0.165 μM in PBS, and 200 μL was added to each glass-mounted cell monolayer. Incubation time was 40 min then cells were washed in PBS and mounted onto slides using ProLong Gold Antifade reagent (Molecular Probes). Slides were imaged on an Olympus FV1000-xyconfocal microscope at 40× magnification in 0.1 μm sections at excitation wavelength of 663 and emission wavelength of 690 in channel 1 and ex/em λ494/521 in channel 2. Sections were rendered in 3D using velocity software (Improvision, Walton, Mass.).

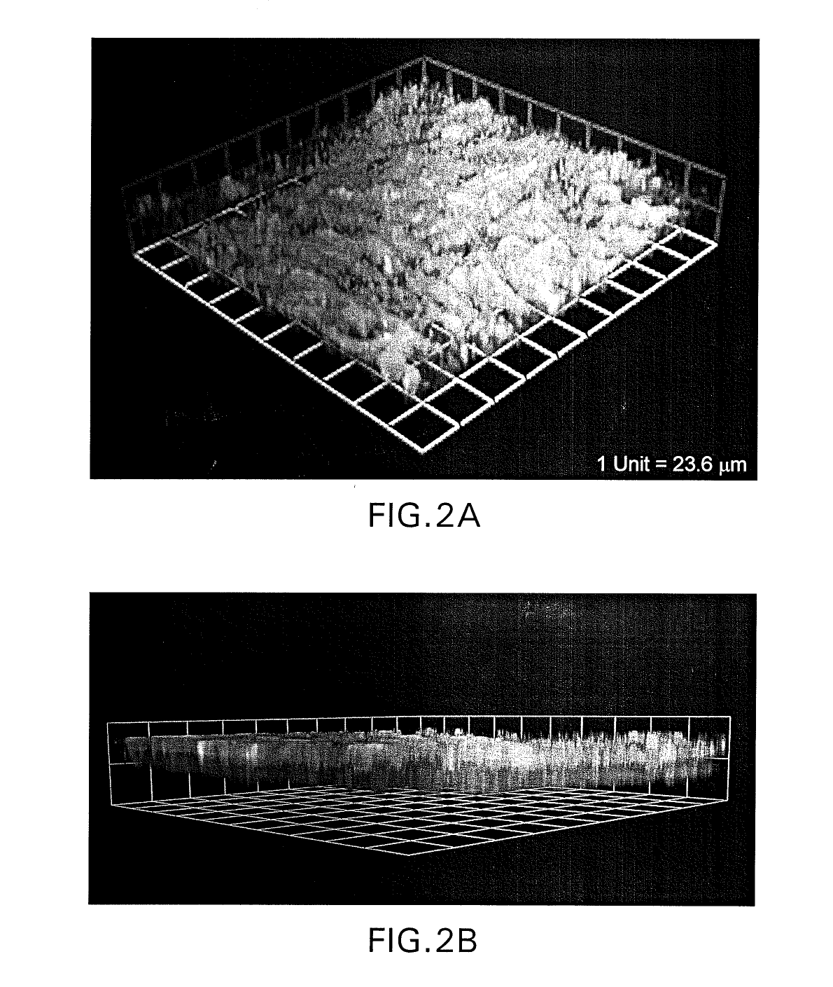

This examples shows that positively charged copolymers can interact with the endothelial glycocalyx using fluorescence microscopy on BLMVEC monolayers exposed to 40P-FITC-TMA-Cl. (FIG. 2; a clear line is visible between the extracellular polymer and the internal actin cytoskeleton, showing that the polymer is able to interact with the glycocalyx at the cell surface).

### Example 4

### Albumin Diffusion Study

The diffusion of labeled albumin across confluent monolayers is a model for protein extravasation through the endothelial barrier and serves as a measure of barrier integrity.

BLMVEC were subcultured on Costar Transwell chambers (polycarbonate membranes, 0.4 μm pore sizes, 1.12 cm² growth area) at a density of 2.5×10⁵ cells/cm². Before seeding, chambers were treated with bovine gelatin and fibronectin and experiments were performed 7-10 days after plating. On the day of the experiment, warm PRF-MII was added to a custom block chamber designed to hold Transwell chambers. Cell media containing 1% TMR-BSA in place of bovine serum albumin was added to the ablumenal chamber of the Transwell directly onto BLMVEC monolayers, with or without polymer solution at a final volume of 0.5 mL and TMR-BSA concentration of 0.5 mg/mL. The ablumenal chamber was sampled once per hour replacing media, and the TMR-BSA presence was detected on a 96-well plate reader at ex/em λ541/572. A permeability diffusion coefficient was derived using the following equation:

P_{DA}=(ΔC×Aᵥₒₗ)/(A×t×L_{conc})

where P_{DA} is the diffusive permeability coefficient (cm/s), ΔC is the change in the tracer concentration in the ablumenal chamber, Aᵥₒₗ is the ablumenal chamber volume (mL), A is the monolayer surface area (cm²), t is time (s), and L_{conc} is the luminal concentration of tracer (mg/mL). Post-experimentation cells were fixed in formalin and stained with Ladd Multiple Stain (Ladd Research Industries; Burlington, Vt.) to ensure confluent and intact monolayers. (N=3 replicates per group.)

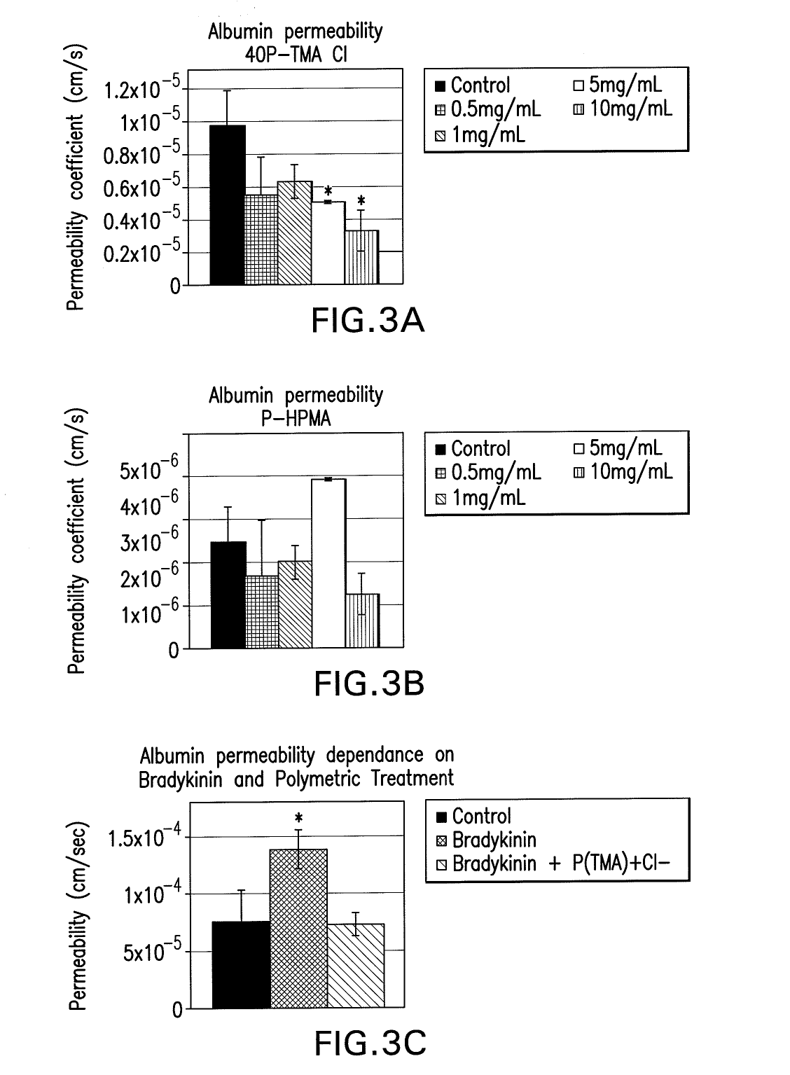

The ability of polymers to reduce protein flux across the endothelium was tested on confluent endothelial monolayers subjected to increasing concentrations of 40P-TMA-Cl. The rate of labeled albumin flux through the monolayers was recorded as a permeability coefficient (PDA). Monolayers treated with 5 and 10 mg/mL concentrations of 40P-TMA-Cl had a significant reduction in the permeability coefficient compared to control monolayers receiving no treatment (FIG. 3). When P-HPMA was used in place of 40P-TMA-Cl, no statistical difference was seen among any of the groups (FIG. 3B). Since there is no pressure upon the cell monolayer to force copolymer into the glycocalyx, P-HPMA has not effect on P_{DA} thus it is clear that ionic contacts are important to bring the copolymer into contact with the cell surface to reduce protein flux from the apical to basal sides of the cell monolayers.

To test the ability of polymers to decrease P_{DA} under inflammatory conditions, endothelial monolayers were exposed to the inflammatory agonist bradykinin (1 μM) during experimentation. Addition of bradykinin to cells increased the albumin permeability coefficient by nearly twofold after one hour (FIG. 3C). After one subsequent hour of monolayer exposure to bradykinin and 40P-TMA-Cl, the permeability coefficient was reduced to that of control cells receiving neither polymer nor bradykinin. This is likely due to the ability of 40P-TMA-Cl to intercalate into the glycocalyx and block serum proteins from moving from one side of the barrier to the other, thus enhancing the passive function of the glycocalyx.

### Example 5

### Hydraulic Conductivity Measurements

Previous work has shown the influence of hydrostatic pressure on the flux of fluid from the capillary lumen to the surrounding tissue (Dull et al., Am J Physiol Lunc Cell Mol Physiol 292:L1452-8, 2007; Florian et al., Circ Res 93(10):e136-42, 2003). The intercalation of polymer into the glycocalyx is believed to reduce mechanotransduction, which occurs when glycoproteins on the cell surface undergo perturbations due to hemodynamic forces. Biomimetic polymer is believed to dampen signaling to the actin cytoskeleton, which contacts transmembrane glycoproteins, focal adhesions and adherens junctions, all of which regulate fluid flux across the capillary. It has been previously shown that pressure increases across endothelial monolayer to the basolateral side and that removal of key components of the glycocalyx abolishes the pressure-induced increase in hydraulic conductivity (Lₚ) (Dull et al., Am J Physiol Lunc Cell Mol Physiol 292:L1452-8, 2007), thus providing evidence that the glycocalyx plays an active role in mechanotransduction.

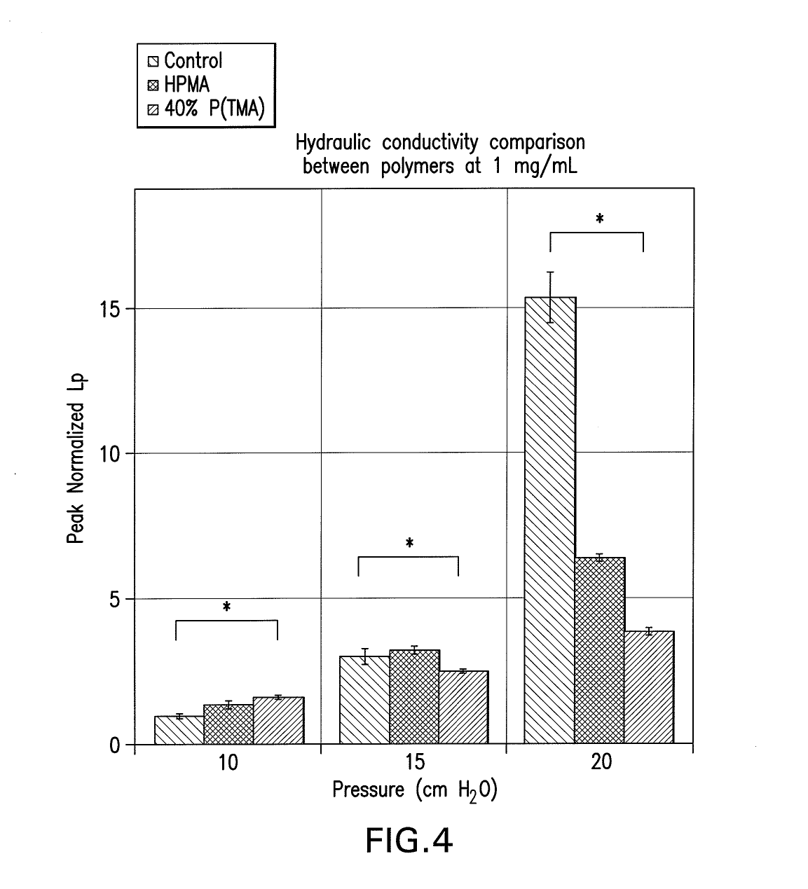

The system used to measure to volumetric flux rate (Jᵥ) and hydraulic conductivity (Lₚ) is described in Hubert et al., Microvasc Res 71:135-40, 2006. Briefly, Snapwell filters housing confluent monolayers of BLMVEC were inserted into custom permeability chambers. To measure Lₚ the inflow port of the cell culture chamber was connected by Silastic tubing to a pressure manifold filled with MII. Fluid flow was tracked by the location of an air bubble as it passed through a length of clear borosilicate capillary tubing. A digital video camera was mounted above the tubing and streamed continuous digital images of the capillary tubes and air bubbles to the data acquisition virtual instrument written in LabView (National Instruments, Austin Tex.). Cell monolayers were allowed to equilibrate for 1 h under a hydrostatic pressure of 1 cm H₂O. After 1 h, either a 1 mg/mL solution of 40P-TMA-Cl, P-HPMA, or MII (control group) only were applied to the chamber housing BLMVEC monolayers. Cells were washed with MII after 30 min and a pressure of 10 cm H₂O was applied. Water pressure was increased by 5 cm H₂O every hour and Lₚ data were constitutively collected every 5 s. A schematic for the experiment process is given in FIG. 4. Post experiment, Snapwell chambers were fixed in formalin and stained with Ladd Multiple Stain to ensure confluency. All monolayers included in data analysis were 100% confluent at the end of each experiment (N=5-7 chambers per group.)

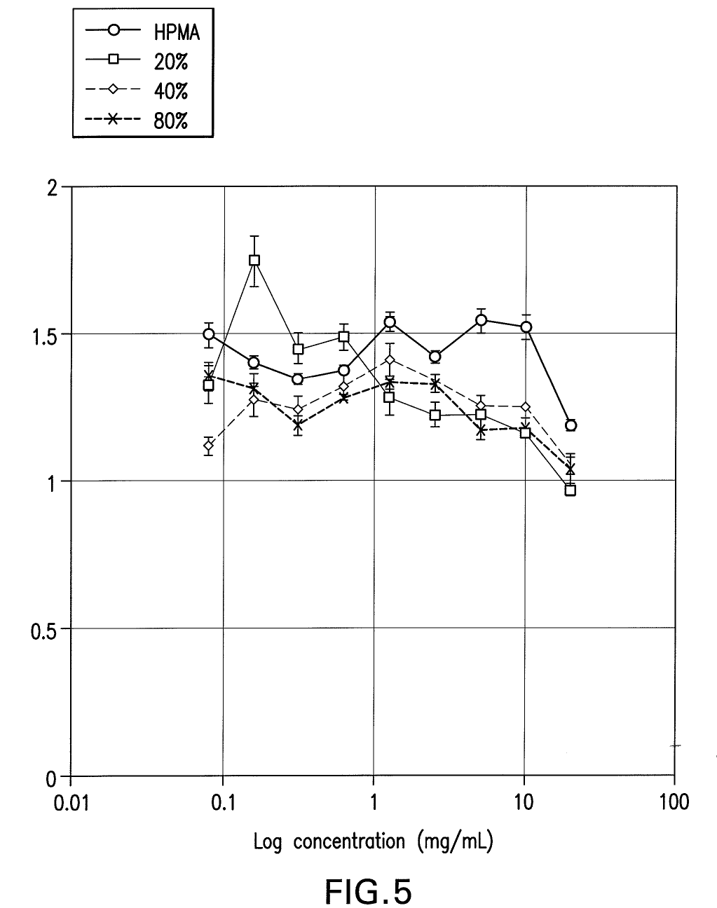

Copolymer solutions were applied to cell monolayers prior to Lₚ experimentation. A schematic for the experiment protocol is given in FIG. 4. Lₚ values were normalized to the average of the Lₚ values acquired during the last 5 min of the 10 cm H₂O pressure interval in order to facilitate comparison between experiments. Peak normalized Lₚ values are the average of the Lₚ values acquired during the last 5 min of a given interval. Control measurements are recorded at 10 cm H₂O then monolayers are treated with polymer. In control monolayers that are not exposed to polymer, normalized Lₚ values for 15 and 20 cm H₂O are 3 and 15 times higher than those at 10 cm H₂O, respectively. Decreases in Lₚ across endothelial monolayers in the presence of P-TMA is evidence of a dampening of mechanical signaling. See also FIG. 5.

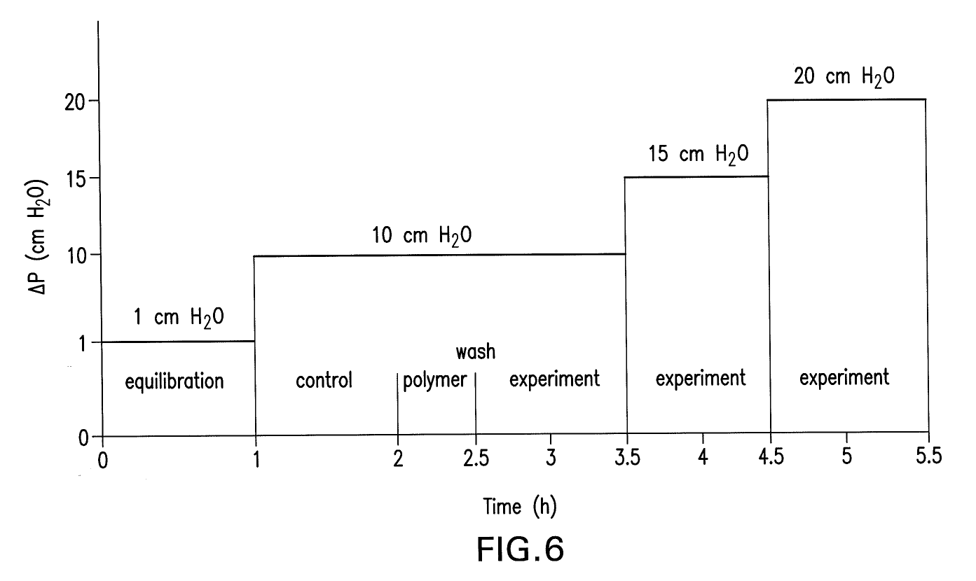

FIG. 6 compares cells receiving no polymer treatment to those receiving 40P-TMA-Cl and P-HPMA at a concentration of 1 mg/mL. Treatment with 40P-TMA-Cl decreased Lₚ by 76% compared to controls after cells had been subjected to pressures of 20 cm H₂O. Increasing the polymer concentration by 10-fold further reduced Lₚ by 20% but when compared to controls, percent Lₚ reduction was 76% and 81% for concentrations of 1 mg/mL and 10 mg/mL, respectively. These figures show compelling evidence for the ability of infusible polymers targeted to lung endothelium to enhance the function of the glycocalyx by reducing hydraulic conductivity across endothelial monolayers, thereby attenuating a major component of vascular inflammation. The decrease in hydraulic conductivity suggest polymer intercalation into the glycocalyx, which can either enhance the passive role of the glycocalyx by creating a thicker barrier for fluid to pass through or attenuate the active role of mechanotransduction by limiting the motion of cell surface glycoproteins.

It will be apparent to those skilled in the art that various modifications and variations can be made in the present invention without departing from the scope or spirit of the invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A composition comprising a copolymer having the following formula: 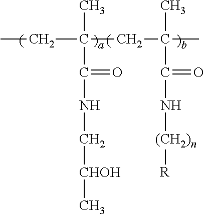
where R is NH₂ or ⁺N(R¹)₃X⁻, wherein each R¹ is, independently of the others a lower alkyl, X is F⁻, Cl⁻, Br⁻, I⁻, SO₄²⁻, CO₂⁻, OH⁻, or CH₃CO₂⁻; n is an integer of from 1 to 6; and
a and b are integers from 1 to 2000.

2. The composition of claim 1, wherein the copolymer has the formula:

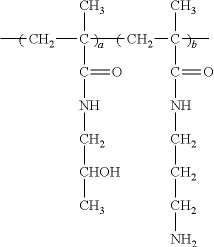

3. The composition of claim 2, wherein the mole percent of N-(3-aminopropyl)methacrylamide residues in the copolymer is from about 10 to about 50 mol %.

4. The composition of claim 2, wherein the mole percent of N-(3-aminopropyl)methacrylamide residues in the copolymer is about 20 mol %.

5. The composition of claim 2, wherein the mole percent of N-(3-aminopropyl)methacrylamide residues in the copolymer is about 40 mol %.

6. The composition of claim 2, wherein the copolymer further comprises a residue of 5-3-(methacryloylaminopropyl)thioureidyl fluorescein.

7. The composition of claim 1, wherein the copolymer has the formula:

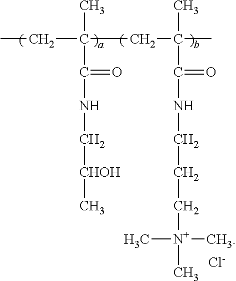

8. The composition of claim 7, wherein the mole percent of methylacrylamidopropyl trimethyl ammonium chloride residues in the copolymer is from about 10 to about 50 mol %.

9. The composition of claim 7, wherein the mole percent of methylacrylamidopropyl trimethyl ammonium chloride residues in the copolymer is about 20 mol %.

10. The composition of claim 7, wherein the mole percent of methylacrylamidopropyl trimethyl ammonium chloride residues in the copolymer is about 40 mol %.

11. The composition of claim 7, wherein the copolymer further comprises a residue of 5-3-(methacryloylaminopropyl)thioureidyl fluorescein.

12. The composition of claim 1, wherein the copolymer further comprises a residue of 5-3-(methacryloylaminopropyl)thioureidyl fluorescein.

13. The composition of claim 1, further comprising a bioactive agent.

14. A method of treating a patient diagnosed with a cardiopulmonary disease comprising contacting lung microvasculature of the patient with an effective amount of a copolymer of claim 1.

15. The method of claim 14, wherein the cardiopulmonary disease is pulmonary edema.

16. The method of claim 15, wherein the pulmonary edema is caused by acute lung injury or acute respiratory distress syndrome.

17. The method of claim 14, wherein the patient is a burn victim.

18. The method of claim 14, wherein the cardiopulmonary disease is related to cardiopulmonary bypass surgery, solid organ transplant, or major vascular surgery.

19. A method of treating a patient diagnosed with a cardiopulmonary disease comprising contacting lung microvasculature of the patient with an effective amount of a copolymer of N-2-hydroxypropyl methacrylamide and methylacrylamidopropyl quaternary ammonium halide.
